(19) United States
(12) Patent Application Publication
(10) Pub. No.: US 2010/0277312 A1
(43) Pub. Date: Nov. 4, 2010

(54) IN-LINE HIGH-THROUGHPUT CONTRABAND DETECTION SYSTEM

(76) Inventors: Peter Michael Edic, Albany, NY (US); Mark E. Vermilyea, Niskayuna, NY (US); John Eric Tkaczyk, Delanson, NY (US); Pierfrancesco Landolfi, Newark, CA (US); Geoffrey Harding, Hamburg (DE); Sondre Skatter, Oakland, CA (US); Matthew Allen Merzbacher, Oakland, CA (US); Helmut Rudolf Strecker, Hamburg (DE); Cameron John Ritchie, Newark, CA (US)

Correspondence Address: Patent Docket Department;Armstrong Teasdale LLP, 7700 Forsyth Boulevard, Suite 1800, St. Louis, MO, 63105, US

(21) Appl. No.: 12/032,116

(22) Filed: Feb. 15, 2008

Related U.S. Application Data
(60) Provisional application No. 60/891,145, filed on Feb. 22, 2007.

(51) Int. Cl. G08B 21/00; A61B 6/03; G01N 23/04; G01R 33/20; G06K 9/00

(52) U.S. Cl. 340/540; 378/4; 378/57; 324/307; 382/143; 378/70; 378/95

(57) ABSTRACT

A contraband detection system includes a first contraband detection apparatus designed to perform a first scan on an object and a second contraband detection apparatus positioned in-line with the first contraband detection apparatus to perform a second scan on the object. A computer is included in the contraband detection system and is programmed to cause the first contraband detection apparatus to perform the first scan and acquire object data therefrom. The computer is further programmed to identify one or more regions of interest (ROI) in the object based on the object data, cause the second contraband detection apparatus to perform the second scan on the one or more identified ROIs, and acquire object data from the second scan.

### CROSS-REFERENCE TO RELATED APPLICATION

The present application is a non-provisional of, and claims priority to, U.S. Provisional Application Ser. No. 60/891,145, filed Feb. 22, 2007.

### BACKGROUND OF THE INVENTION

Embodiments of the invention relate generally to contraband detection systems and, more particularly, to a method and apparatus for detecting contraband using combined imaging technologies.

In recent years, the detection of contraband, such as explosives, being transported in luggage and taken onto various means of transportation has become increasingly important. To meet the increased need for such detection, advanced Explosives Detection Systems (EDSs) have been developed that can not only detect suspicious articles being carried in the luggage but can also determine whether or not the articles contain explosive materials.

These detection systems, at a minimum, include computed tomography (CT) machines that are capable of acquiring mass and density information (as well as materials specific information, such as an effective atomic number) on items within luggage. Although object density is an important quantity, surrogates such as “CT number” or “CT value” which represent a linear transformation of the density data may be used as the quantity indicative of a threat. Although density is described in the embodiments below, all quantities are applicable and can be used interchangeably. Moreover, the features such as mass, density, and effective atomic number embody derived quantities such as statistical moments, texture, etc. of such quantities. To acquire more detailed and highly selective information on luggage being scanned, explosives detection devices based on other technologies such as quadrupole resonance (QR), trace detection, or x-ray diffraction (XRD) can be employed in combination with the CT system. These devices provide complementary information relative to the data from the CT system, thereby improving the overall detection performance of the EDS. That is, the complementary information gained from the systems and detection techniques ancillary to CT can provide higher detection sensitivity with reduced false alarms as compared to CT data alone, thus resulting in less manual or follow-on inspection needed to clear the alarms and preventing inspection system backup. Collectively, multiple technologies are required to satisfy (at the very least) minimum detection requirements for the whole range of explosives as specified by the Transportation Security Administration (TSA). Typically, the explosives detection devices are manufactured as stand-alone units, which are connected by the baggage handling system within an airport; the information provided by each system may or may not be combined optimally for overall threat assessment.

While existing EDSs that combine various scanning and detection technologies have been adequate to date, challenging requirements exist for future generations of explosives detection systems for baggage. Increases in the number of traveling passengers, increasing variance in explosive materials, and possible modifications to the concept of operations due to emerging threats will increase the demand for EDSs with improved throughput to accommodate the increased volume of baggage and require more sensitive/specific means for explosives detection. Moreover, next generation explosives detection systems will be required to meet threat detection standards commensurate with the Transportation Security Administration's current and future requirements (e.g., the TSA 2010 requirements), which may include, for example, single digit false alarm rates, throughput of at least 1000 bags per hour, ease of integration of new systems into the baggage handling system, and 99.5% availability.

To meet future TSA mandated detections standards, EDSs will require improved imaging performance and the combination of data from multiple sensors. The combination of presently employed third-generation CT scanners with technologies such as XRD, for example, can meet such standards; however, existing combinations of these technologies cannot meet the increased throughput rates that will be required. That is, typically, the CT system and the XRD system are stand-alone systems, which limits combined throughput capability of baggage scanning. Since the XRD system is typically located separate from the CT system, the XRD system requires an integrated pre-screener to acquire radiographic data that facilitates registration of a particular baggage item to previously acquired CT data. Registration of the baggage item with respect to previously acquired CT data allows for proper identification of suspected threat positions (i.e., regions of interest (ROIs)) in the baggage item, which is needed for XRD interrogation. Once the baggage item has been registered and the ROIs identified, the baggage item is moved into the XRD system and the x-ray source and collimator/detector arrangement in the system are mechanically positioned to direct x-rays that traverse the ROIs. While the above procedure allows for increased accuracy in XRD scanning, such registration and identification of the suspected threat position, along with the mechanical positioning of the x-ray source and collimator/detector arrangement in the XRD system, can lead to increased scanning time and greatly reduce baggage scanning rates.

Therefore, it would be desirable to design an apparatus and method for increasing throughput in an EDS while maintaining explosives detection at high sensitivity and simultaneously at low false alarm rates. It would also be desirable to have increased efficiency in identifying regions of interest in the baggage via CT data that represent a small fraction of the total baggage area and to control a follow-up imaging system where this ROI can be interrogated by highly selective follow-up imaging techniques with minimum adjustments or maintenance thereto.

### BRIEF DESCRIPTION OF THE INVENTION

Embodiments of the invention are directed to a method and apparatus for contraband detection that overcome the aforementioned challenges. A contraband detection system is disclosed that includes a first contraband detection apparatus positioned in-line with a second contraband detection apparatus and integrated therewith to increase scanning throughput capability for baggage or other objects of interest. Regions of interest (ROIs) in the baggage are identified by the first contraband detection apparatus and information on the ROIs is sent to the second contraband detection apparatus to facilitate subsequent scanning instructions thereto. The ROIs may be comprised of specific points in the baggage item or include the entire baggage item.

According to an aspect of the invention, a contraband detection system includes a first contraband detection apparatus to perform a first scan on an object and a second contraband detection apparatus positioned in-line with the first contraband detection apparatus to receive the object after passing through the first contraband detection apparatus and perform a second scan on the object. The contraband detection system also includes a computer connected to the first and second detection apparatuses programmed to cause the first contraband detection apparatus to perform the first scan, acquire object data from the first scan, and identify one or more regions of interest (ROI) in the object based on the object data, the one or more ROIs comprising one of a portion of the object or the entire object. The computer is further programmed to cause the second contraband detection apparatus to perform the second scan on the one or more identified ROIs, and acquire object data from the second scan.

According to another aspect of the invention, a method for detecting contraband includes the steps of performing a first scan on an object in a first scanning system to acquire a first set of data and identifying at least one region of interest (ROI) in the object based on the acquired first set of data, the at least one ROI comprising one of a portion of the object or the entire object. The method also includes the steps of passing the object to a second scanning system positioned in-line with the first scanning system and performing a second scan on the object to acquire a second set of complementary data, the second scan comprising the at least one ROI.

According to yet another aspect of the invention, an integrated imaging system for detecting contraband includes a first scanning system designed to convey and scan a baggage item to acquire scan data and a second scanning system positioned in-line with the first scanning system to receive the baggage item therefrom and designed to scan the baggage item to acquire complementary scan data. The integrated imaging system for detecting contraband also includes a processing unit connected to the first and second scanning systems programmed to cause the first scanning system to scan the baggage item to acquire the scan data, identify one or more regions of interest (ROI) in the baggage item based on the received scan data, and generate a desired scanning pattern for the second scanning system for the one or more identified ROIs. The processing unit is further programmed to cause the second scanning system to scan the baggage item using the desired scanning pattern to acquire the complementary scan data.

These and other advantages and features will be more readily understood from the following detailed description of preferred embodiments of the invention that is provided in connection with the accompanying drawings.

### BRIEF DESCRIPTION OF THE DRAWINGS

FIG. 1 illustrates a contraband detection system according to an embodiment of the invention.

FIG. 2 is a pictorial view of a CT imaging system for use with the system of FIG. 1.

FIG. 3 is a block schematic diagram of the system illustrated in FIG. 2.

FIG. 4 is a schematic diagram of an x-ray diffraction system for use with the system of FIG. 1.

FIG. 5 is illustrative of a stationary distributed x-ray source and diffraction detector for use with the system of FIG. 4.

FIG. 6 a schematic of the Explosives Detection System of FIG. 1, illustrating generation and modification of a Threat State for a baggage item.

FIG. 7 illustrates a contraband detection system according to another embodiment of the invention.

### DETAILED DESCRIPTION OF PREFERRED EMBODIMENTS

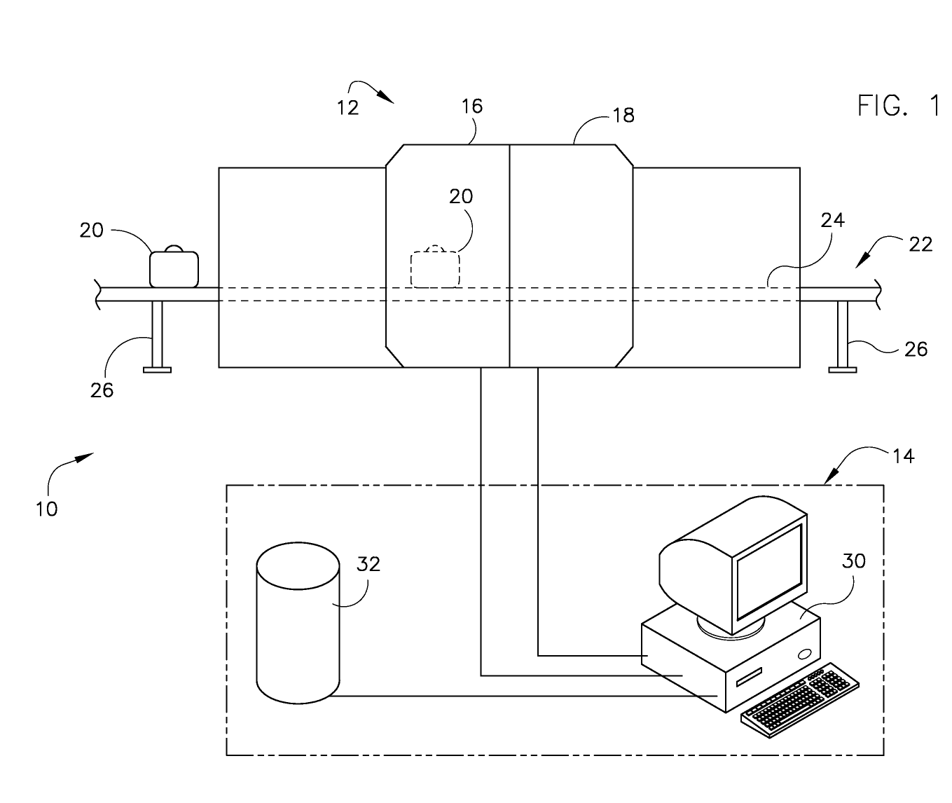

Referring to FIG. 1, a contraband detection system 10 (i.e., explosives detection system (EDS) 10) is shown. Although specific mention of an explosives detection system 10 is provided in preferred embodiments described below, other contraband detection system such as for narcotics, knives, guns, etc. are contemplated. EDS 10 includes a scanning subsystem 12 and a computer subsystem 14. The scanning subsystem 12 includes a first scanner system 16 (i.e., first contraband/explosives detection apparatus) and a second scanner system 18 (i.e., second contraband/explosives detection apparatus). The first and second scanner systems 16, 18 can include, but are not limited to, any of a known combination of scanning systems, such as a computed tomography (CT) scanner and an x-ray diffraction (XRD) scanner, a CT scanner and a quadrupole resonance (QR) scanner, or a CT scanner and any other contraband scanner (e.g., trace detection system). As shown in FIG. 1, second scanner system 18 is positioned in-line with first scanner system 16, to receive luggage, baggage, or other objects of interest 20 directly therefrom. While first and second scanner systems 16, 18 are shown as a physically integrated EDS 10, the system may be separate entities placed in close proximity to one another. In such an arrangement, however, the systems must maintain registration of the spatial coordinate system to facilitate overall system scanning operations. Furthermore, as will be explained below, the data acquired from both systems is also integrated/shared to increase the throughput of baggage 20 through the EDS 10 and the overall threat detection performance. Although both scanning systems 16, 18 can be configured to scan the entire baggage item 20 and the data retrospectively evaluated for overall threat assessment, the queuing of subsequent scanning systems by data acquired from the first scanning system 16 facilitates overall system throughput by identifying suspicious regions of interest in the baggage item 20.

A conveyor system 22 is also provided and includes a conveyor belt 24 supported by a structure 26 to automatically and continuously pass packages or baggage pieces 20 through passageways extending through both the first and second scanner systems 16, 18 such that a throughput of baggage items 20 for scanning in first scanner system 16 and second scanner system 18 is provided. Baggage items 20 are fed through first and second scanner systems 16, 18 by conveyor belt 24 while imaging data is acquired, and the conveyor belt 24 moves the baggage items 20 through the scanners 16, 18 in a controlled and continuous manner. As a result, postal inspectors, baggage handlers, and other security personnel may non-invasively inspect the contents of baggage 20 for explosives, knives, guns, narcotics, contraband, etc. Conveyor belt 24 passes baggage items 20 in a manner that preserves the relative position of baggage item 20 and contents therein, such that second scanner system 18 examines locations within baggage items 20 at a coordinate location identified/flagged by first scanner system 16, as explained in detail below.

Referring still to FIG. 1, the computer subsystem 14 of EDS 10 includes a computer 30 and an electronic database 32, which is connected to the computer 30. Computer 30 is connected to both of first and second scanner systems 16, 18 to receive data therefrom and send data thereto, as will be explained in greater detail below. It is envisioned that computer subsystem 14 controls operation of both the first and second scanner systems 16, 18, as is shown in FIG. 1; however, it is also contemplated that separate computers be associated with each imaging device and be connected via a network (not shown) to provide data to computer subsystem 14.

In one embodiment, and as shown and described in detail herebelow, first scanner system of EDS 10 can comprise a CT scanner 16 and second scanner system of EDS 10 can comprise an XRD scanner 18; however, it is envisioned that other embodiments of EDS 10 may incorporate additional types of contraband/explosives detection apparatuses, such as a quadrupole resonance scanner, trace detection system, or other contraband scanner. Additionally, while CT scanner 16 of the EDS 10 is described here below as a “third generation” CT system, it will be appreciated by those skilled in the art that the embodiments of the invention are equally applicable with other CT systems, such as those that may incorporate stationary and/or distributed x-ray sources.

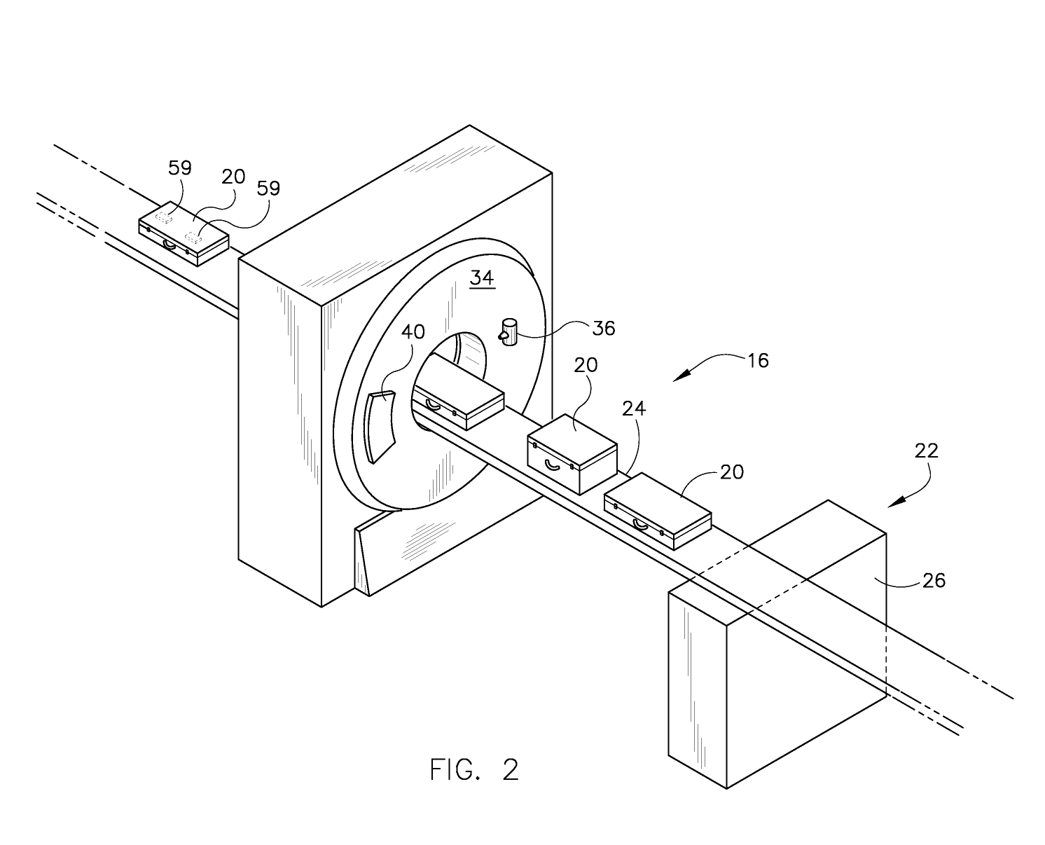

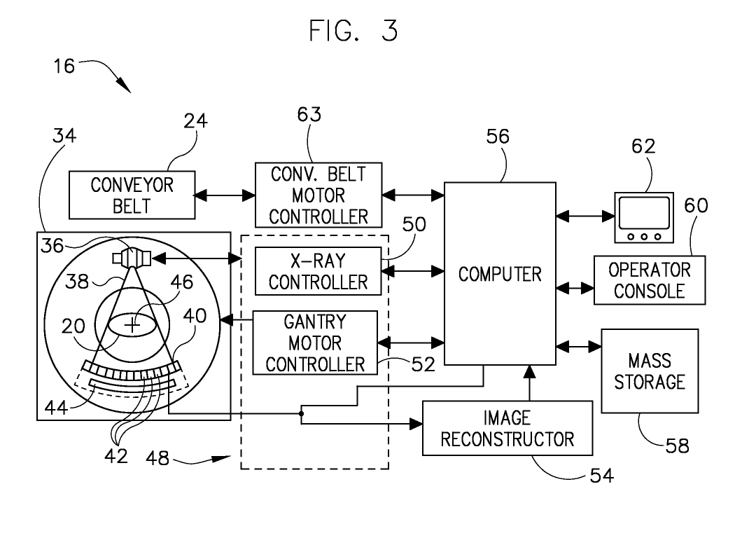

Referring now to FIGS. 2 and 3, an isolated view of the computed tomography (CT) scanner 16 is shown as including a gantry 34 representative of a “third generation” CT scanner. Gantry 34 has an x-ray source 36 that projects a beam of x-rays 38 toward a detector assembly 40 on the opposite side of the gantry 34. As shown in FIG. 3, detector assembly 40 is formed by a plurality of detectors 42 and a data acquisition system (DAS) 44. The plurality of detectors 42 sense the projected x-rays that pass through the volume containing baggage item 20, and DAS 44 converts the data to digital signals for subsequent processing. Each detector 42 produces an analog electrical signal that represents the intensity of an impinging x-ray beam from which the integral of beam attenuation along that finite-width line within baggage item 20 can be measured.

During a scan to acquire x-ray projection data, gantry 34 and the components mounted thereon rotate about a center of rotation 46. The projection data corresponds to processed x-ray intensity measurements to represent line integrals of linear attenuation coefficient within the scanned items 20, which is well-known in the art. Rotation of gantry 34 and the operation of x-ray source 36 are governed by a control mechanism 48 of CT system 16. Control mechanism 48 includes an x-ray controller 50 that provides power and timing signals to an x-ray source 36 and a gantry motor controller 52 that controls the rotational speed and position of gantry 34. An image reconstructor 54 receives sampled and digitized x-ray data from DAS 44 and performs high-speed reconstruction thereon to output “CT data.” The CT data, in the form of reconstructed images, is applied as an input to a computer 56, which stores the images in a mass storage device 58.

As image reconstructor 54 and computer 56 are incrementally reconstructing “slices” of CT data by any of a number of mathematical algorithms and techniques (e.g., conventional filtered back-projection techniques), 2-D segmentation is also being performed on each of the reconstructed slices by computer 56. A 2-D image segmentation technique, such as edge detection, watershed segmentation, level sets, or another known segmentation method, is applied to each reconstructed image slice to identify regions in the slice that may be indicative of the presence of an explosive material. That is, each image slice reconstructed from the CT data represents the mass and density characteristics of that “slice” of the baggage item 20. Regions of interest (ROI) 59 (shown in FIG. 2) in the baggage 20 having mass and/or density characteristics that may possibly correspond to a known explosive material can be identified by way of the 2-D segmentation. As will be described below, these ROIs 59 are identified for further examination in the XRD system to better quantify the likelihood of an explosive material being present in the baggage item 20. Although 2D segmentation techniques are mentioned, limited-volume 3D segmentation techniques are also contemplated.

Computer 56 also receives commands and scanning parameters from an operator via console 60 that has some form of operator interface, such as a keyboard, mouse, voice activated controller, or any other suitable input apparatus. An associated display 62 allows the operator to observe the reconstructed image and other data from computer 56. The operator-supplied commands and parameters are used by computer 56 to provide control signals and information to DAS 44, x-ray controller 50 and gantry motor controller 52. In addition, computer 56 can operate a conveyor belt motor controller 63 which controls conveyor belt 24 to position and pass baggage items 20 in and through gantry 34. As set forth above, computer 56 can be specific to CT system 16 or can be embodied as computer subsystem 14 of the EDS 10 shown in FIG. 1. Additionally, image reconstructor 54 may be embodied with the CT system 16, or a remote device.

It is also envisioned that CT scanner 16 may comprise an energy sensitive (ES), multi-energy (ME), and/or dual-energy (DE) CT imaging system. An ESCT imaging system, by providing energy-sensitive detection of x-rays, acquires sufficient information to determine material specific properties of items within baggage 20 by way of a determination of the effective atomic number of materials present in the baggage. In one embodiment, detectors 42 are designed to directly convert x-ray energy to electrical signals containing energy discriminatory or photon count data. That is, detectors 42 detect each x-ray photon reaching each detector 42, and DAS 44 records the photon energy according to energy deposition in the detector. The detectors 42 are therefore composed of a material capable of the direct conversion of x-ray energy, such as Cadmium Zinc Telluride (CZT) or another suitable material, to provide such energy discrimination capability.

In another embodiment of an ESCT imaging system, x-ray controller 50 functions to vary the operating voltage of x-ray source 36 to provide energy discriminating capability to CT system 16. That is, x-ray controller 50 is configured to control a generator (not shown) to apply different peak kilovoltage (kVp) levels to x-ray source 36, which changes the peak energy and spectrum of the incident photons comprising the emitted x-ray beams 38. Thus, CT system 16 may acquire projections sequentially at varying energy levels. The detected signals from the two energy levels, generally characterized as high and low, provide sufficient information to determine the material specific properties of items within baggage item 20 by way of the determination of the effective atomic number of those items. Although two specific embodiments of energy sensitive CT systems are provided, any suitable method for acquired energy sensitive projection data and subsequent identification of the effective atomic number distribution within baggage item 20 are suitable substitutes.

It is envisioned that additional aspects of CT system 16 can be modified within the scope of the invention to accommodate increased throughput rates of baggage 20 through the scanner. For example, detectors 42 can be modified to increase the number of rows of detector elements/pixels in each detector, thus increasing the coverage per gantry rotation for each baggage scan. Additionally, the rotational speed of gantry 34 can be varied (i.e., increased) to allow for a higher throughput of baggage items 20 through CT system 16.

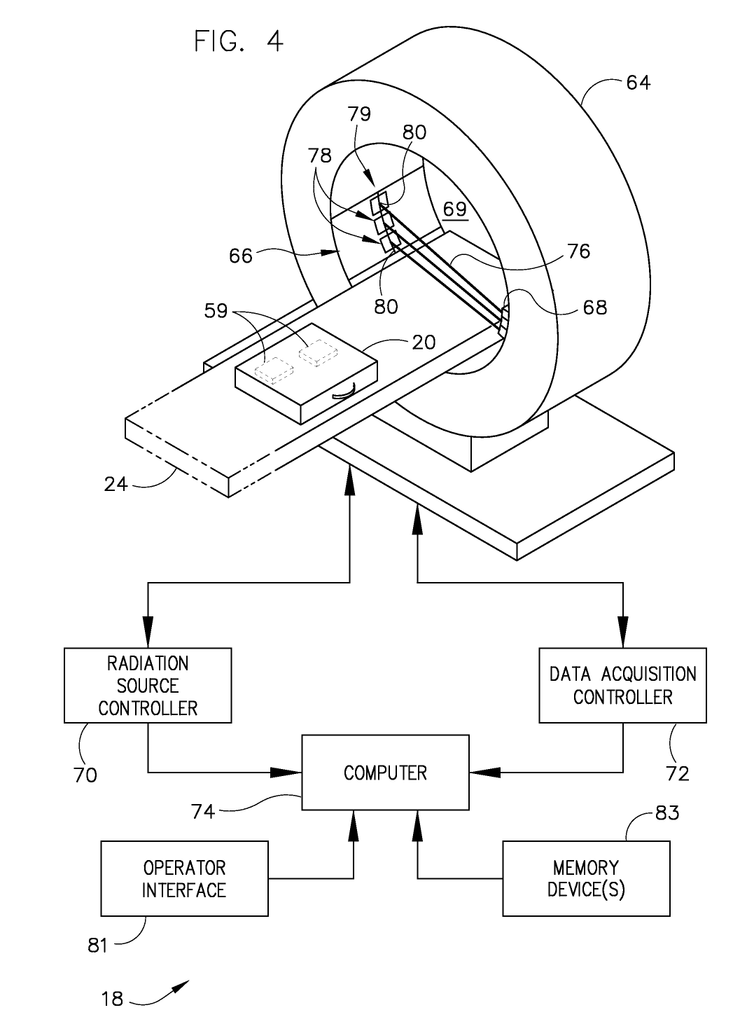

Referring now to FIG. 4, an isolated view of x-ray diffraction (XRD) system 18 is illustrated. The XRD system 18 comprises a gantry 64 having positioned thereon a stationary and distributed source of x-ray radiation 66 and one or more stationary detectors 68 that are fixed on gantry 64. The XRD system 18 is configured to receive conveyor belt 24 through a bore 69 in gantry 64 to allow for passage of baggage items 20 therethrough that are passed on from CT scanner 16. As described in greater detail below, data acquired from CT scanner 16 for identifying one or more ROIs 59 in the baggage 20 is used to control the operation of the XRD system 18.

To control operation of distributed x-ray source 66 and detector 68, the XRD system 18 includes a radiation source controller 70 and a data acquisition controller 72, which may both function under the direction of a computer 74. As set forth above, computer 74 can be specific to XRD system 18 or can be embodied as computer subsystem 14 of the EDS 10 shown in FIG. 1. The radiation source controller 70 regulates timing and location for discharges of x-ray radiation 76, which is directed from source locations 78 on the distributed x-ray source 66 toward detectors 68 positioned on an opposite side of gantry 64. The radiation source controller 70 may trigger a cathode module 79 having one or more emitters 80 positioned thereon and at source locations 78 in the distributed x-ray source 66 at each instant in time for acquiring multiple x-ray diffraction data. In certain arrangements, for example, the x-ray radiation source controller 70 may trigger emission of radiation in sequences from different source locations 78 in distributed x-ray source 66, as will be explained in detail below. In addition, although in a preferred embodiment the stationary distributed x-ray source 66 is comprised of multiple field emission devices, the electron beams can be generated from one of many types of electron emitters, such as thermionic cathodes. Moreover, a single electron beam can be generated and steered using electromagnetic or electrostatic fields to generate multiple x-ray source locations, while still maintaining the stationary nature of the distributed source.

The x-rays 76 sent from the distributed x-ray source 66 pass through one or more ROIs 59 in baggage item 20, are diffracted by the specific material present in the ROI 59, and are directed onto the detector 68, which measures the coherent scatter spectra of the x-rays after passing through the ROI 59 to acquire “XRD data.” The coherent scatter spectra of the x-rays may then be processed and compared to a library of known reference spectra for various dangerous substances (i.e., explosives) that can be stored on computer 74. As such, a signature for the molecular structure of a material in the ROI 59 can be analyzed and a determination made to discern if that structure corresponds to a known explosive material. Many such measurements may be collected in an examination sequence, and data acquisition controller 72, which is coupled to detector 68, receives signals from the detector 68 and processes the signals, thus acquiring the XRD data.

Computer 74 generally regulates the operation of the radiation source controller 70 and the data acquisition controller 72. The computer 74 may thus cause radiation source controller 70 to trigger emission of x-ray radiation 76, as well as to coordinate such emissions during imaging sequences defined by the computer 74. The computer 74 also receives data acquired by data acquisition controller 72 and coordinates storage and processing of the data. An operator interface 81 may be integral with the computer 74 and will generally include an operator workstation for initiating imaging sequences, controlling such sequences, and manipulating data acquired during imaging sequences, which can be stored in a memory device 83. Operator interface 81 of XRD system 18 may be combined with the operator console of the CT system 16 (FIG. 1) to provide one common operator interface (not shown).

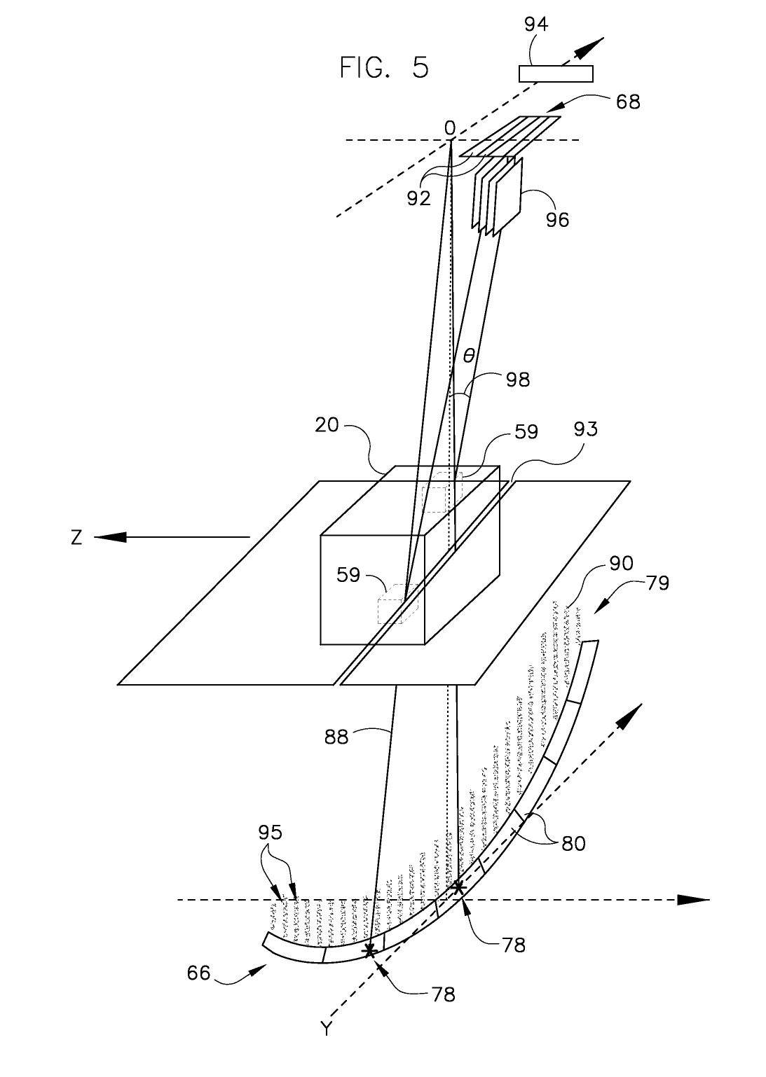

Referring now to FIG. 5, a portion of exemplary distributed x-ray sources 66 of the type that may be employed in the stationary XRD system 18 is shown. The distributed x-ray sources 66 may include multiple cathode modules 79, with each cathode module 79 comprising one or more electron beam emitters 80 that are positioned at source locations 78 and coupled to radiation source controller 70 (shown in FIG. 4) by way of activation connections (not shown). Emitters 80 are triggered by the source controller 70 during operation of the XRD system 18. Emitters 80 are positioned facing an anode (not shown) and, upon triggering by the source controller 70, the emitters 80 emit electron beams toward the anode. Upon striking of the electron beams on the anode, which may, for example, be a tungsten rail or element, a primary beam of x-ray radiation is emitted, as indicated at reference numeral 88. The primary x-ray beams 88 are directed, then, toward a collimator 90, which is generally opaque to the x-ray radiation, but which includes apertures 95. The apertures 95 may be fixed in dimension, or may be adjustable, to permit primary x-ray beams 88 to penetrate through the collimator 90 to form focused, collimated primary x-ray beams. The primary x-ray beams 88 are directed to an imaging volume 93 of the XRD scanner 18, pass through one or more ROIs 59, and are diffracted to impact detector 68 on an opposite side of the XRD scanner 18.

A number of configurations for emitters 80 and/or distributed sources 66 are envisioned. In one embodiment, for example, distributed x-ray source 66 comprises a cold cathode field emitter array that is positioned apart from a stationary anode. As shown in FIG. 5, distributed x-ray source 66 is arcuate in shape so as to be positionable about a portion of the bore 69 (shown in FIG. 4) in XRD scanner 18. Linear distributed x-ray sources can also be employed so as to extend along the imaging plane 93, in the “in-plane direction.” Other materials, configurations, and principles of operations may also be employed for the distributed x-ray source 66.

Referring still to FIG. 5, one or more stationary detectors 68 are oriented along the z-axis (i.e., parallel to the direction of baggage throughput) and each of the detectors 68 is comprised of a plurality of detector elements 92, which receive the radiation emitted by the distributed x-ray source 66 and diffracted by a material in ROI 59. Signal processing circuitry, such as an application specific integrated circuit (ASIC) 94, is associated with each detector 68. Detector elements 92 can be configured to have varying resolution so as to satisfy a particular imaging application. A collimator 96 is positioned adjacent to detectors 68 that allows the detector elements 92 to measure only radiation at a constant scatter angle 98 with respect to the orientation of the primary x-ray beams 88 emitted from the distributed x-ray source 66. In one embodiment, XRD scanner 18 is configured as an “inverse geometry” system in which distributed x-ray source 66 is arcuate in shape and covers a much greater area than detector 68, such as the distributed x-ray source and detector arrangement set forth in U.S. Pat. No. 6,693,988 to Harding et al. It is also envisioned, however, that distributed x-ray source 66 be linear in shape and that detector 68 may comprise alternate configurations.

In one embodiment, detectors 68 are also configured for energy resolution less than 3% at an x-ray photon energy of 60 keV and can be energy sensitive detectors comprised of high-purity germanium, CZT, or other suitable energy sensitive detector technology. Collimators 96 provide the coding of the constant angle diffraction signal resulting from the interaction of the x-ray beam with the baggage 20, allowing measurement of a diffraction signal from a particular region of interest.

As described above, cathode modules 79, and corresponding emitters 80, within distributed x-ray source 66 are independently and individually addressable so that radiation can be triggered from each of the source locations 78 at points in time as needed. The triggering of a particular cathode module 79 and its emitters 80 is determined by the one or more ROIs 59 identified in the baggage item 20 via the CT scanner 16. As set forth above, the ROIs 59 are identified by way of an analysis of the CT data (e.g., 2D segmentation or limited 3D segmentation of reconstructed data) and the mass, density, and/or effective atomic number characteristics in the CT data that may be indicative of an explosive material. These identified ROI(s) 59 within the baggage item 20 is/are then mapped to determine where the ROI 59 lie within the field-of-view 93 of the CT system 16 and XRD system 18.

In selecting activation of a desired emitter 80 at a source location 78 in distributed x-ray source 66, data related to the location of the ROI 59 within the field-of-view 93 are sent to computer 74 (shown in FIG. 4). A desired emitter 80 is then selected/activated based on its proximity to the ROI 59, with the emitter 80 that provides an x-ray beam that traverses ROI 59 being activated. More precisely, an emitter 80 is selected from the plurality of emitters in the cathode module 79 of stationary distributed x-ray source 66 whose resulting primary x-ray beam 88 most overlaps a centroid of the ROI 59. If more than one ROI 59 is identified in the baggage item 20, an activation sequence is determined (by computer 74) in which a plurality of the emitter elements 80 are sequentially activated or queued in a desired activation order, with the selection/activation of each emitter 80 based on the overlap of its primary x-ray beam with a respective ROI 59. The computer 74 queues the activation of emitters 80 based on their association with the ROI 59 and the location of the ROI 59 within baggage item 20 (and field-of-view 93) to optimize a scanning process in the XRD scanner 18 and to achieve a maximum throughput rate of baggage 20 through XRD scanner 18. Beneficially, as no rotation or repositioning of an x-ray source/detector arrangement is required, but only electrical activation of selected emitters 80 in the stationary distributed x-ray source 66, no time delay for x-ray source/detector re-positioning is experienced.

While described above as being individually or sequentially activated, in other configurations, the emitters 80 are addressable in logical groups. For example, pairs or triplets of emitters 80 may be logically “wired” together. Where desired, and as determined by the identified ROI 59, more than one such group of emitters 80 may be triggered concurrently at any instant in time.

Based on the acquired CT data (mass, density, and/or effective atomic number) and XRD data (spectral signature indicative of the molecular structure, noted as “molecular signature), a “Threat Status” for one or more ROI 59 in a particular piece of baggage 20 can be generated. That is, a determination can be made of the probability and/or likelihood of an explosive material being present in the baggage item 20. Toward this end, computer subsystem 14 (shown in FIG. 1) has programmed thereon a common set of threat categories, which in one embodiment can mirror the Transportation Security Administration's categorization of explosives. Each of these threat categories contains information on mass, density, effective atomic number, and molecular signature characteristics that are specific to explosives in that category.

In combining the mass, density, effective atomic number, and molecular signature characteristics obtained in the CT data and XRD data for an identified ROI, a Bayesian Data Fusion Protocol, employing Bayes' law, can be implemented. That is, the risk calculus and determination of a probability/likelihood of contraband/explosives may be characterized by Bayesian probability theory wherein the initial risk values are probabilities of the presence of each type of contraband based on a first type of scan. The probabilities are modified using Bayes' rule, with the initial risk values of the first scan being applied to and combined with risk values ascertained from scanning results of a second type of scan, to output a final risk value that is the combination of probabilities for the given types of contraband/explosives based on the combination of scans. The combination of probabilities, and corresponding final risk value, are output as the Threat Status. Although not described herein, statistical techniques other than those based on Bayesian statistics are contemplated as being useful for combining the data from multiple scanning devices.

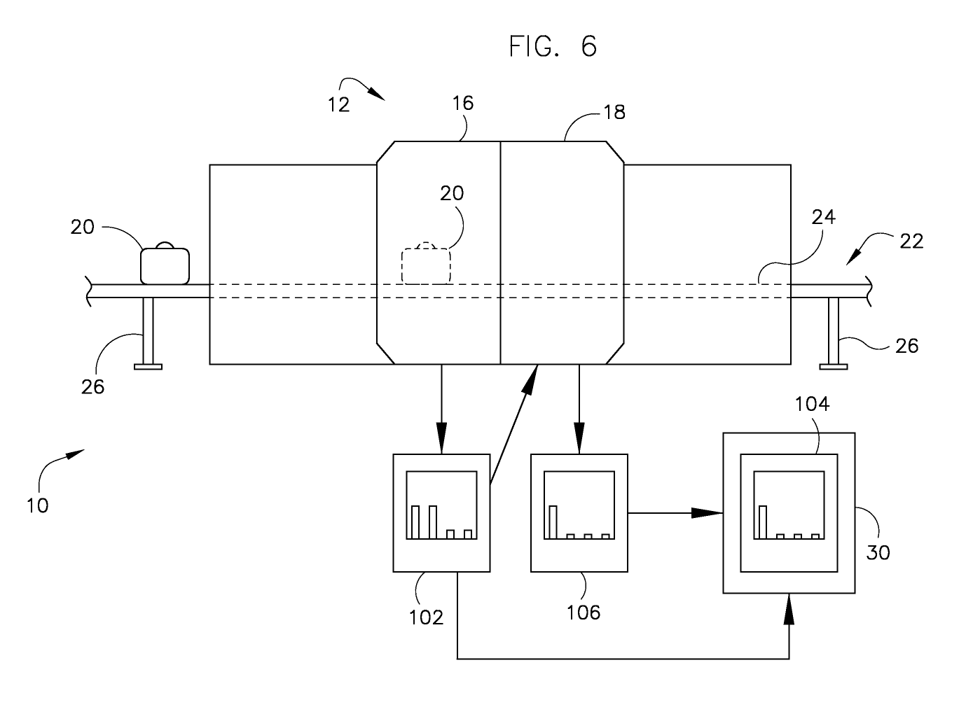

Referring now to FIG. 6, a graphical representation of EDS 10 and the use of a Bayesian Data Fusion Protocol to determine a Threat Status is illustrated. As illustrated in FIG. 6, CT data is acquired for an item of baggage 20, whereby at least one of mass, density, and effective atomic number characteristics for the baggage 20 are determined from the acquired CT data. A preliminary threat state 102 is output for each ROI identified in the baggage item 20. The preliminary threat state 102 includes probabilities that the baggage item 20 includes the various types of contraband/explosives that are included in the pre-defined threat categories. The preliminary threat state 102 can be shown on a display device 104 of the computer 30.

The conveyor belt 24 then moves the baggage item 20 into the XRD scanner 18, which scans any ROI in the baggage item 20, as described in detail above. As illustrated in FIG. 6, the preliminary threat state 102 is sent to the XRD scanner 18, which, based on molecular signatures acquired for materials in the ROI, modifies the preliminary threat state 102 to generate an updated or final threat state 106, depending on the number of scanners/sensors in the system. The final threat state 106 includes a plurality of modified probabilities/likelihoods that the baggage item 20 includes one of the various types of contraband/explosives included in the preliminary threat states. The final threat state 106 can also then be shown on display device 104 of computer 30.

The computer 30 reads the final threat state 106 and, if the total probability of any type of contraband being in the baggage item 20 is above the critical probability for any particular threat category, the computer 30 triggers an alarm to alert an operator of the EDS 10 of the likely presence of contraband/explosives. The alarm could be one of a visual alarm displayed on computer 30, an audio alarm, or a means for extracting the suspect baggage item from the normal stream of baggage.

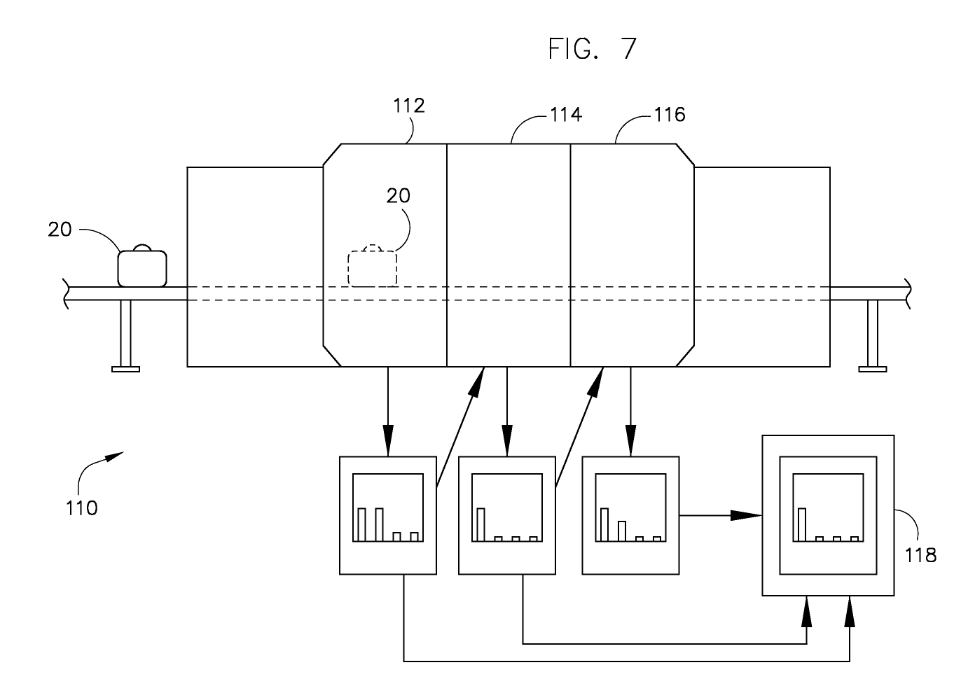

While the above contraband detection system is described as being comprised of first and second contraband detection apparatuses, it is further contemplated that additional scanning devices can be included in the contraband detection system. That is, one or more additional scanning devices can be positioned in-line with the first and second contraband detection apparatuses, and complementary data therefrom can be further combined with the data acquired by the first and second contraband detection apparatuses and integrated therewith. Referring now to FIG. 7, an EDS 110 is shown that includes a first contraband detection apparatus 112, a second contraband detection apparatus 114, and a third contraband detection apparatus 116. The first, second, and third detection apparatuses 112, 114, 116 can include, but are not limited to, any of a known combination of scanning systems, including a computed tomography (CT) scanner, an x-ray diffraction (XRD) scanner, a quadrupole resonance (QR) scanner, and any other contraband scanner (e.g., trace detection system). Object data is acquired for an item of baggage 20 by first contraband detection apparatus 112, such as CT data, whereby at least one of mass, density, and effective atomic number characteristics for the baggage 20 are determined. One or more ROIs are identified in the baggage item 20 based on this data and this data is passed onto the second contraband detection apparatus 114, which then scans any ROIs in the baggage item 20, as described in detail above. Another type of object data (e.g., molecular signature characteristics) is thus acquired for the ROIs by second contraband detection apparatus 114. The baggage item 20 is then passed onto third contraband detection apparatus 116 and yet additional complementary object data for the ROIs is acquired. Such data can, for example, comprise nuclear quadrupole resonance (NQR) data that identifies atoms whose nuclei have a nuclear quadrupole moment, which is measured by way of a radio frequency NQR response from the ROIs.

The object data acquired by first, second, and third detection apparatuses 112, 114, 116 (and any additional scanning devices integrated into EDS 110) is assessed/combined by computer 118, as set forth in detail above with respect to FIG. 6. The combined object data allows for the generation of probabilities/likelihoods that the baggage item 20 includes any of various types of contraband/explosives therein and for the generation of threat states, as set forth above.

A technical contribution for the disclosed method and apparatus is that it provides for a computer implemented method and apparatus that increases throughput scanning capability for baggage or other objects of interest by identifying regions of interest in the baggage and providing scanning instructions to a stationary x-ray diffraction system.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Furthermore, while explosives detection techniques are discussed above, the invention encompasses other types of contraband, such as concealed weapons and narcotics. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. A contraband detection system, comprising:
a first contraband detection apparatus to perform a first scan on an object;
a second contraband detection apparatus positioned in-line with the first contraband detection apparatus to receive the object after passing through the first contraband detection apparatus and perform a second scan on the object; and
a computer connected to the first and second detection apparatuses programmed to:
cause the first contraband detection apparatus to perform the first scan;
acquire object data from the first scan;
identify one or more regions of interest (ROI) in the object based on the object data, the one or more ROIs comprising one of a portion of the object or the entire object;
cause the second contraband detection apparatus to perform the second scan on the one or more identified ROIs; and
acquire object data from the second scan.

2. The contraband detection system of claim 1, wherein the computer is further programmed to combine the object data from the first and second scans to detect the presence of contraband in the object and, if contraband is detected, assign a threat level and generate an alert comprising one of an audible or visual alert.

3. The contraband detection system of claim 1, wherein the first contraband detection apparatus comprises a computed tomography (CT) system.

4. The contraband detection system of claim 1, wherein the second contraband detection apparatus comprises a non-translational x-ray diffraction (XRD) system, the non-translational XRD system including a stationary distributed x-ray source to emit a primary x-ray beam therefrom and a stationary detector to receive x-rays diffracted by the ROI, the distributed x-ray source having a plurality of x-ray source locations therein.

5. The contraband detection system of claim 4, wherein the computer is programmed to activate one or more desired x-ray source locations in the distributed x-ray source based on the identified ROI, each of the one or more desired x-ray source locations emitting an x-ray beam that most overlaps a centroid of the ROI as compared to x-ray beams emitted from other x-ray source locations in the distributed x-ray source.

6. The contraband detection system of claim 5, wherein, when the one or more ROI comprises a plurality of ROIs, the computer is programmed to activate a plurality of desired source locations in the distributed x-ray source in one of a queued activation pattern and a concurrent activation pattern.

7. The contraband detection system of claim 4, wherein the stationary distributed x-ray source comprises an arc-shaped distributed x-ray source configured to emit an inverse fan-beam of x-rays.

8. The contraband detection system of claim 4, wherein the stationary detector comprises:
a collimator configured to pass diffracted x-rays having a constant scatter angle with respect to the primary x-ray beam;
an energy sensitive sensor element to receive the passed x-rays and convert the passed x-rays to corresponding electrical signals; and
an application specific integrated circuit (ASIC) to condition the electrical signals.

9. The contraband detection system of claim 1, wherein the computer is further programmed to:
define a plurality of threat categories;
determine a first threat probability for each threat category based on the first scan;
determine a second threat probability for each threat category based on the second scan;
combine the first threat probability and the second threat probability; and
output a combined threat probability for each threat category to determine the probability of contraband being present in the object.

10. The contraband detection system of claim 1, wherein the acquired object data comprises at least two of density, mass, effective atomic number characteristics, and molecular signature characteristics of the object.

11. The contraband detection system of claim 1, wherein the second contraband detection apparatus comprises one of a quadrupole resonance (QR) system and a trace detection system.

12. The contraband detection system of claim 1, comprising one or more additional contraband detection apparatuses configured to perform an additional scan on the one or more identified ROIs to acquire additional complementary object data.

13. A method for detecting contraband, comprising the steps of:
performing a first scan on an object in a first scanning system to acquire a first set of data;
identifying at least one region of interest (ROI) in the object based on the acquired first set of data, the at least one ROI comprising one of a portion of the object or the entire object;
passing the object to a second scanning system positioned in-line with the first scanning system; and
performing a second scan on the object to acquire a second set of complementary data, the second scan comprising the at least one ROI.

14. The method of claim 13, comprising the step of combining the first and second sets of acquired data to determine the presence of contraband in the object, the presence of contraband being determined by at least two of density, mass, effective atomic number characteristics, and molecular signature characteristics obtained from the first and second sets of complementary data.

15. The method of claim 13, wherein the step of performing a first scan comprises scanning an object with x-rays in a computed tomography (CT) scanning system to acquire one or more of mass, density, and effective atomic number.

16. The method of claim 15, wherein the step of identifying the ROI comprises:
reconstructing the acquired CT data for each of a plurality of 2D slices; and
identifying reconstructed CT data in one of a 2D slice or a limited 3D volume formed from the plurality of 2D slices having at least one of a density, mass, and effective atomic number characteristic indicative of contraband.

17. The method of claim 13, wherein the step of performing a second scan comprises generating an x-ray beam from a stationary distributed x-ray source in an x-ray diffraction (XRD) scanning system to scan the ROI and acquire XRD data.

18. The method of claim 17, wherein the stationary distributed x-ray source comprises one or more cathode modules, each cathode module containing electron emitter elements comprising one or more field emitter elements, one or more thermionic elements, or one or more steered solitary electron beam sources.

19. The method of claim 18, wherein generating an x-ray beam comprises:
selecting one or more electron emitter elements from a plurality of emitter elements in the stationary distributed x-ray source whose primary radiation beams most overlap a centroid of the ROI; and
electronically activating the one or more selected emitter elements by way of activation connections to the one or more selected emitter elements.

20. The method of claim 19, wherein the step of activating the one or more selected emitter element comprises activating a plurality of specified emitter elements in one of a queued activation pattern and a concurrent activation pattern based on the identified ROI.

21. An integrated imaging system for detecting contraband, comprising:
a first scanning system designed to convey and scan a baggage item to acquire scan data;
a second scanning system positioned in-line with the first scanning system to receive the baggage item therefrom and designed to scan the baggage item to acquire complementary scan data; and
a processing unit connected to the first and second scanning systems, the processing unit programmed to:
cause the first scanning system to scan the baggage item to acquire the scan data;
identify one or more regions of interest (ROI) in the baggage item based on the received scan data, the one or more ROIs comprising one of a portion of the baggage item or the entire baggage item;
generate a desired scanning pattern for the second scanning system for the one or more identified ROIs; and
cause the second scanning system to scan the baggage item using the desired scanning pattern to acquire the complementary scan data.

22. The integrated imaging system of claim 21, wherein the processing unit is programmed to combine the scan data from the first scanning system and the complementary scan data from the second scanning system to detect the presence of contraband in the baggage item and, if contraband is detected, assign a threat level and generate an alert comprising one of an audible or visual alert.

23. The integrated imaging system of claim 21, wherein the first scanning system comprises a computed tomography (CT) scanner including a gantry having a bore designed to receive the conveyed baggage item, the gantry having an x-ray source and an x-ray detector disposed thereon to emit x-rays toward the baggage item and receive x-rays attenuated by the baggage item, respectively, to acquire CT projection data; and
wherein the second scanning system comprises an x-ray diffraction (XRD) system positioned in-line with the CT scanner and having a stationary distributed x-ray source and a stationary detector to emit a primary beam of x-rays toward the one or more ROIs in the baggage item and receive x-rays diffracted by the one or more ROIs, respectively, to acquire XRD data.

24. The integrated imaging system of claim 23, wherein the processing unit is further programmed to:
receive the CT projection data;
determine the one or more ROI based on one or more of density, mass, and effective atomic number characteristics contained in the CT projection data; and
activate a desired x-ray source location in the stationary distributed x-ray source based on a location of the one or more ROIs within the baggage item, the location of the one or more ROIs being closest to the primary beam of x-rays generated by the x-ray source location.

25. The integrated imaging system of claim 23, wherein the processing unit is further programmed to determine the probability of contraband being present in the object by way of meaningfully combining the CT data and the XRD data.
